(19) United States
(12) Patent Application Publication
(10) Pub. No.: US 2014/0228584 A1
(43) Pub. Date: Aug. 14, 2014

(54) Process for the production of a dioxolane compound from crude glycerol including a liquid-liquid extraction step

(71) Applicants: Edson Rodrigues, Pirassununga (SP) (BR); Arthur Malheiro, Araraquara (SP) (BR)

(72) Inventors: Edson Rodrigues, Pirassununga (SP) (BR); Arthur Malheiro, Araraquara (SP) (BR)

(73) Assignee: RHODIA POLIAMIDA E ESPECIALIDADES LTDA, Säo Paulo (BR)

(21) Appl. No.: 14/347,249

(22) PCT Filed: Sep. 30, 2011
(86) PCT No.: PCT/IB2011/002300

§ 371 Date: Mar. 26, 2014

(51) Int. Cl. C07D 317/20

(52) U.S. Cl. CPC C07D 317/20
USPC 549/453

(57) ABSTRACT

A process for the production of dioxolane compounds of formula (I), wherein R1 and R2 independently represent hydrogen or an alkyl chain from 1 to 10 carbon atoms; R3 and R4 independently represent hydrogen, an alkyl chain from 1 to 5 carbon atoms or an alkyl chain from 1 to 5 carbon atoms endowed with one or more hydroxyl groups. Such process comprises successively a reaction step between crude glycerol and aldehyde or ketone and a liquid-liquid extraction step with an extracting ketonic solvent corresponding to formula (II) with A and B independently being an alkyl chain with 2 to 10 carbon atoms, or phenyl, A and B not comprising hydroxyl functions.

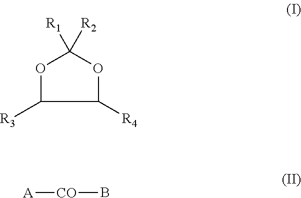

The present invention concerns a process for the production of dioxolane compounds comprising a step of reaction between crude glycerol and aldehydes or ketones, and a step of liquid-liquid extraction.

### BACKGROUND ART

### Crude Glycerol

Crude glycerol is a by-product of the process to obtain biodiesel, namely a transesterification of vegetable raw material, such as natural oils or animal fat, with lower alcohols.

Such a transesterification process is the method most often employed to enable the use of vegetable oils (for instance coconut, soy, castor, sunflower, peanut) and animal fat as fuel, for instance in the presence of a homogeneous or heterogeneous alkaline catalyst.

From the transesterification reaction in a basic or acid medium, one obtains a monoalkyd ester—which is the biodiesel fuel—and glycerol.

A general equation for that reaction would be:

triglyceride+3 alkyl alcohol→glycerol+3 fatty acid alkyl ester

The monoalkyd ester and the glycerol formed in the transesterification reaction are substantially immiscible and are separated by decantation at the end of the reaction.

Very commonly, in that process to obtain biodiesel, the neutralization of the transesterification product with hydrochloric acid gives origin to a sodium chloride contamination solubilized in the decanted glycerin. The presence of sodium chloride accelerates corrosion in stainless steel equipment.

This raw glycerol, byproduct of the transesterification reaction, has low purity and contains, among several contaminants, fatty acids, fatty acid salts, inorganic salts, inorganic acids, inorganic bases, water, lower alcohols, mono, di and triglycerides, esters of fatty acids with lower alcohols, transesterification catalyst residue, etc. To enable the use of this raw glycerol, the traditional path has been the removal of its contaminants with several purification steps, to obtain a purer product commonly referred to as blond glycerin, which is then bi-distilled, to reach high purity. It is obviously a long and costly process when compared to the direct use of the crude glycerol.

### Dioxolane and Process to Obtain it

Dioxolanes, in the sense utilized herein, are a group of organic compounds containing the 1,3-dioxolane ring, known to be used in several applications, such as pharmaceutical actives, chemical intermediates and solvents. The particular use as solvent is interesting as it is less harmful than traditional solvents, with similar performance.

The preparation of a dioxolane compound by way of reacting glycerol and a ketone or an aldehyde is generally known, for instance as in the following publications: R. J. Fessenden 85 J. F. Fessenden, Organic Chemistry, 2nd edition, page 524, 1982 and T. W. Greene, Protective Groups in Organic Chemistry, John Wiley 86 Sons, 1981. No mention to the use of crude glycerol and the effects of the contaminants upon the reaction is mentioned.

### THE INVENTION

The present invention concerns a process for the production of dioxolane compounds comprising at least two steps, namely:

A—the reaction between crude glycerol and aldehyde or ketone;

B—a liquid-liquid extraction with an extracting ketonic solvent.

The reaction between crude glycerol and aldehyde or ketone leads to the formation of at least two layers in the reaction vessel. The top layer, where most of the resulting dioxolane is present, also comprises aldehyde or ketone, water, glycerol, and a chloride content typically up to 1.0% wt. For increasing the purity of dioxolane, especially by decreasing such undesired content of chlorides, which would otherwise promote corrosion in any equipment utilized in further operations with the resulting dioxolane, the present invention proposes to subject said top layer to a liquid-liquid extraction with an extracting solvent. According to the invention, certain ketonic solvents were surprisingly found to be far more effective than others in the liquid-liquid extraction step aimed at lowering the chloride content.

The lower layer or layers obtained from the reaction step, comprising mainly glycerol, fatty salts, sodium chloride and water, are for instance treated to recycle the glycerol to the reaction vessel or purged. The treatment of those lower layers from the reaction step are not an integrant part of the invention.

More particularly, the invention concerns a process that comprises at least two steps, one step being the reaction between crude glycerol and aldehyde or ketone, aimed at obtaining a dioxolane compound corresponding to formula (I) below

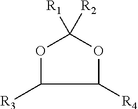

where:
R1 and R2 independently represent hydrogen or an alkyl chain from 1 to 10 carbon atoms, more particularly an alkyl chain from 1 to 5 carbon atoms, for instance methyl, ethyl, propyl, isopropyl, butyl, isobutyl, pentyl and isopentyl.
R3 and R4 independently represent hydrogen, an alkyl chain from 1 to 5 carbon atoms or an alkyl chain from 1 to 5 carbon atoms which is endowed with one or more hydroxyl groups.

According to the present invention, in the reaction between crude glycerol and aldehyde, one of the groups R1 and R2 is hydrogen while in the reaction between glycerol and ketone, the groups R1 and R2 do not represent hydrogen. R1 and/or R2 are particularly chosen from methyl, ethyl, propyl, isopropyl, butyl, isobutyl, pentyl and isopentyl.

According to the process of the invention, a following step to the reaction step, is a liquid-liquid extraction step, the extracting solvent(s) employed corresponding to formula (II) A-CO-B with A and B independently being an alkyl chain with 2 to 10 carbon atoms, or phenyl, A and B not comprising hydroxyl functions.

Without excluding any other, adequate ketones for the reaction step with glycerol are acetone, cyclohexanone, methyl cyclohexanone, methyl cyclopentanone, methyl isobutyl ketone, 4-hydroxy-4-methyl-2-pentanone, 2-butanone, 3-butanone, diisobutyl ketone, 4-methyl-3-penten-2-one, 2-nonanone, 2-pentanone, 3-methyl-2-butanone and 1-phenylethanone and mixtures thereof. A preferred ketone is acetone.

Without excluding any other, adequate aldehydes for the reaction step with glycerol are formaldehyde, acetaldehyde, 2-ethylhexanal and furfuraldehyde and mixture thereof.

In a particular embodiment of the invention, it is possible to utilize one or more ketones and/or one or more aldehydes, preferably selected among ketones and aldehydes listed above, to react with the crude glycerol.

According to the invention, crude glycerol advantageously reacts with ketone and most advantageously with acetone.

Without excluding any other, adequate extracting solvents for the liquid-liquid extraction step are ketonic solvents preferably selected among MIBK (methyl isobutyl ketone), DIBK (diisobutyl ketone) and acetophenone, alone or in combination.

The dioxolane compounds obtained by the invention are acetals or ketals.

The acetals are obtained by nucleophilic addition of an alcohol to an aldehyde in acidic conditions, followed by elimination of water. The ketals are obtained by the same type of reaction performed with ketones.

Without excluding any other, particular 1,3-dioxolanes obtained by the reaction step are 2-hydrocarbyl-1,3-dioxolane-4-methanol, for instance:
2,2-dimethyl-1,3-dioxolane-4-methanol
2,2-diisobutyl-1,3-dioxolane-4-methanol
2-isobutyl-2-methyl-1,3-dioxolane-4-methanol
2-butyl-2-ethyl-1,3-dioxolane-4-methanol
2-phenyl-1,3-dioxolane-4-methanol

A preferred dioxolane resulting from the reaction step of the invention between glycerol and acetone is 2,2-dimethyl-1,3-dioxolane-4-methanol, also known as solketal.

Particular embodiments of the reaction step comprised in the process of the invention, not excluding any other, are properly performed according to one or more of the following parameters:
temperature: between 10 and 60° C., preferably between 20 and 45° C.;
pressure: atmospheric;
duration: preferably 30 min to 5 hours, most preferably 1 to 2 hours;
catalyst: acid, such as sulfuric acid, metanesulfonic acid, xylenesulfonic, acetic acid, adequate amounts of catalyst are preferentially comprised between 0.2 to 1% weight in relation to the weight of glycerol.
solvent: none or organic solvents like heterocyclic and aromatic organic compounds. Among heterocyclic and aromatic organic compounds, furan compounds are preferred, for example Tetrahydrofuran (THF) and 2-Methyltetrahydrofuran (2MeTHF).
alkalinity: between 0.04 and 10 mg KOH/g, in particular from 0.08 to 1.8 mg KOH/g of glycerol
crude glycerol: as such, preferably obtained as a by-product of the process for producing biodiesel, namely a transesterification of vegetable raw material, such as natural oils or animal fat, with lower alcohols. Crude glycerol advantageously contains glycerol from 40 to 95%, preferably from 75 to 90% by weight, water from 1 to 15%, preferably from 5 to 15% by weight, inorganic salts, especially chlorides, from 1 to 15%, preferably from 4 to 15% by weight and other organic impurities like free fatty acids, salts of fatty acids, esters, sulfur compounds, proteins and minerals.

Crude glycerol is advantageously previously dehydrated, for instance under vacuum, at 70-80° C., so that the content of water is lower than 5% by weight.
a molar ratio aldehyde:glycerol or ketone:glycerol from 3:1 to 20:1, preferably from 4:1 to 15:1, most preferably from 4:1 to 12:1.

In a particular embodiment, the invention concerns a process for the production of a dioxolane compound of formula I characterized by the fact that it comprises the following steps:
a)—reacting crude glycerol and ketone or aldehyde;
b)—allowing appearance of a top light phase and one or more lower heavy phases;
c)—separating said top light phase from other lower heavy phases;
d)—subjecting the separated light phase to a liquid-liquid extraction by contacting said separated light phase with said extracting ketonic solvent of formula II,
e)—allowing appearance of an extracted liquid phase and a raffinate liquid phase;
f)—recovering the raffinate liquid phase;
g)—recovering the extracted liquid phase containing the desired dioxolane compound.

Step a) is carried out in a reaction vessel according to the operating conditions given above. A dehydration of the crude glycerol is preferentially performed prior to step a.

Step b) is adequately performed at 10-60° C., preferably at 20-45° C. for at least one hour.

Step c) is performed by any manner known to the person skilled in the art, for instance decantation, filtration or centrifugation.

Step d) of the invention process which consists in a liquid-liquid extraction may be accomplished using crosscurrent mode or countercurrent mode. In the crosscurrent mode, the extraction equipment can be usually an agitated tank. One or more tank(s) can be used. Solvent is first added to the light phase, the content is mixed, settled and then separated. Single stage extraction or more than one stage can be required. For countercurrent mode, mixer-settlers or columns can be employed. In a particular embodiment, step d is carried out so that the weight ratio between the extracting ketonic solvent and said top light phase ranges from 0.1 to 10, preferably from 0.1 to 5, particularly from 0.5 to 2, more particularly from 0.5 to 1.5.

According to step e) of the invention process, said extracted liquid phase is the upper phase while said raffinate liquid phase is the bottom phase.

Said raffinate liquid phase recovered after step I) of the invention process, contains mainly glycerol, chlorides, especially sodium chloride, and water.

This raffinate phase is for instance treated for recycling the glycerol to the reaction vessel.

Said extracted liquid phase containing the desired dioxolane compound recovered after step g) of the invention process also contains said ketonic solvent and the excess of ketone or aldehyde which does not react in step a). Said extracted liquid phase is preferably separated by distillation. One or several distillation operations may be carried out. A first distillation operation is advantageously carried out for separating said ketonic solvent and the excess of ketone or aldehyde and a second subsequent distillation operation is advantageously carried out for purifying said desired dioxolane compounds. According to a preferred embodiment consisting in using acetone as reagent in step a) and MIBK as solvent, the recovery of acetone and MIBK present in the extracted phase is performed with an atmospheric distillation column at temperature in the range of 60 to 150° C. Most of the water in the extract phase is generally removed by distillation during the recovery of MIBK. A subsequent distillation operation is preferably performed under vacuum (for example at a pressure comprised in the range 1.3*10³−2.7*10³ Pa) at temperature in the range of 110-120° C. for purifying the desired dioxolane compound.

A neutralization step of the top light phase with alkali, for instance sodium hydroxide, followed by filtration, may optionally be performed between steps c and d, or between steps f and g. Adequate amounts of neutralizing alkali are chosen so that the reaction medium reaches an alkalinity between 0.1 and 0.5 mg KOH/g, in particular from 0.2 to 0.3 mgKOH/g of glycerol.

The extracted phase may optionally be subjected to contact with basic resin, activated carbon or a silica system before step g), followed by separation and withdrawal of solids, to further lower level of chlorides, especially sodium chloride.

### EXAMPLE

The following example is given only as a particular embodiment of the invention, in no way imposing limitations beyond the contents of the claims presented further on.

Crude glycerol corresponding to the composition below was used as starting material for the reaction step comprised in the invention (percentages in weight):
glycerol 84.2%
chloride 4.3%
water 5.7%
impurities 5.8%

Acetone was used to react with the crude glycerol.

Molar ratio acetone:glycerol was 4.8:1 (corresponding weight ratio 2.54:1).

The catalyst was sulfuric acid, 0.6% in weight with relation to the weight of glycerol.

The reaction was performed at 400C for 2 hours.

Separation of phases was allowed for 1 hour at 35-40° C.

Two layers were formed, and the top light layer was separated by decantation. The composition (% wt) of this top layer was:
glycerol 5.9%
acetone 60.7%
dioxolane (solketal) 26.0%
water 5.4%
sodium chloride 0.23%
impurities 1.81%

The liquid-liquid extraction step was performed with several commercial solvents tested in addition and mixture to this top layer, in three different solvent:top phase weight ratios: 0.5, 1.0 and 1.5.

Two liquid phases (upper and bottom phases) are obtained after the liquid-liquid extraction with MIBK, DIBK and acetophenone while a bottom solid phase is obtained with isopropanol, isobutanol and octanol. Diacetone alcohol does not lead to the formation of two distinct phases.

The extracted phase (upper phase), obtained by liquid-liquid extraction with MIBK, DIBK and acetophenone, is rich in solketal, acetone and solvent. It is separated by means of several distillation operations.

### Results

Table I below shows the reduction of the chloride content verified after the liquid-liquid extraction with the several tested solvents. The three last solvents, MIBK, DIBK and acetophenone correspond to the invention solvents, and have a remarkable superior performance in removing chlorides from the top layer. With such ketonic solvents, the level of chloride in the extracted liquid phase (upper phase) is inside the acceptable range for stainless steel equipment.

TABLE I

| | Ratio | | |
|---|---|---|---|
| | solvent:top layer | | |
| | 0.5 | 1.0 | 1.5 |
| | % reduction of | | |
| SOLVENT | chloride content | | |
| Isopropanol | 30.6 | 32.6 | 44.3 |
| Isobutanol | 35.2 | 49.0 | 54.4 |
| Octanol | 17.4 | 30.9 | 37.1 |
| Diacetone alcohol | 1.8 | 3.7 | 15.8 |
| MIBK | 90.2 | 93.7 | 97.9 |
| DIBK | 96.6 | 98.1 | 97.7 |
| Acetophenone | 74.1 | 87.9 | 93.0 |

It is understood that with the aid of the information presented herein, the person skilled in the art may deduce the invention to practice in ways not expressly described, but performing substantially the same functions to reach substantially the same results, those equivalent embodiments being encompassed by the following claims.

1. A process for the production of a dioxolane compound of formula (I): 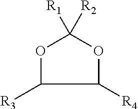
wherein:
R1 and R2 independently represent hydrogen or an alkyl chain from 1 to 10 carbon atoms, and
R3 and R4 independently represent hydrogen, an alkyl chain from 1 to 5 carbon atoms or an alkyl chain from 1 to 5 carbon atoms endowed with one or more hydroxyl groups;
said process comprising successively carrying out:
a reaction step between crude glycerol and an aldehyde or a ketone, wherein when said reaction is between said aldehyde and said crude glycerol, one of said groups R1 and R2 is hydrogen, while when said reaction is between said crude glycerol and said ketone, said groups R1 and R2 do not represent hydrogen; and
a liquid-liquid extraction step with an extracting ketonic solvent corresponding to formula II:
A-CO-B
with A and B independently being an alkyl chain with 2 to 10 carbon atoms, or phenyl, and with A and B not comprising hydroxyl functions.

2. The process according to claim 1, wherein at least one of R1 and R2 is selected from the group consisting of methyl, ethyl, propyl, isopropyl, butyl, isobutyl, pentyl, and isopentyl.

3. The process according to claim 1, wherein said ketonic solvent for said liquid-liquid extraction step is selected from the group consisting of methyl isobutyl ketone (MIBK), diisobutyl ketone (DIBK), acetophenone, and combination thereof.

4. The process according to claim 1, wherein said ketone in said reaction step is at least one ketone selected from the group consisting of acetone, cyclohexanone, methyl cyclohexanone, methyl cyclopentanone, methyl isobutyl ketone, 4-hydroxy-4-methyl-2-pentanone, 2-butanone, 3 -butanone, diisobutyl ketone, 4-methyl-3 -penten-2-one, 2-nonanone, 2-pentanone, 3 -methyl-2-butanone, and 1 -phenylethanone.

5. The process according to claim 1, wherein said aldehyde in said reaction step is one or more ^{of} at least one aldehyde selected from the group consisting of formaldehyde, acetaldehyde, 2-ethylhexanaL and furfuraldehyde.

6. The process according to claim 1, wherein said dioxolane compound obtained by said reaction step is a 2-hydrocarbyl-1,3-dioxolane-4-methanol compound.

7. The process according to claim 1, comprising the following steps:
a)—reacting said crude glycerol and said ketone or said aldehyde;
b)—allowing appearance of a top light phase and one or more lower heavy phases;
c—separating said top light phase from said one or more lower heavy phases;
d)—subjecting the separated top light phase to said liquid-liquid extraction by contacting said separated top light phase with said extracting ketonic solvent of formula (II);
e)—allowing appearance of an extracted liquid phase and a raffinate liquid phase;
f)—recovering said raffinate liquid phase; and
g)—recovering said extracted liquid phase containing the desired dioxolane compound.

8. The process according to claim 7, wherein said step c) is performed by decantation, filtration or centrifugation.

9. The process according to claim 7, wherein a dehydration of the crude glycerol is performed prior to said step a).

10. The process according to claim 7, wherein said step d) is carried out so that the weight ratio between said extracting ketonic solvent and said separated top light phase ranges from 0.1 to 10.

11. The process according to claim 7, wherein a neutralization step of the top light phase with an alkali, followed by filtration, is performed between steps c) and d) or between steps f) and g).

12. The process according to claim 1, wherein said crude glycerol comprises from 40% to 95% by weight of glycerol.

13. The process according to claim 1, wherein said crude glycerol comprises from 1 to 15% by weight of chlorides.

14. The process according to claim 1, wherein said reaction step forms at least two layers in a reaction vessel, said top layer comprising said dioxolane compound and a chloride content up to 1.0% by weight, and wherein said top layer is subjected to said liquid-liquid extraction with said extracting ketonic solvent to lower the chloride content.

15. The process according to claim 7, wherein said extracted liquid phase is an upper phase, and wherein said raffinate liquid phase is a bottom phase.

16. The process according to claim 7, wherein after being recovered after step g), said extracted liquid phase containing the desired dioxolane compound is further separated by one or more distillations to purify said dioxolane compound.

17. The process according to claim 6, wherein said 2-hydrocarbyl-1,3-dioxolane-4-methanol compound is selected from the group consisting of 2,2-dimethyl-1,3-dioxolane-4-methanol, 2,2-diisobutyl-1,3-dioxolane-4-methanol, 2-isobutyl-2-methyl-1,3-dioxolane-4-methanol, 2-butyl-2-ethyl-1,3-dioxolane-4-methanol, and 2-phenyl-1,3-dioxolane-4-methanol.
